(19) United States
(12) Patent Application Publication
(10) Pub. No.: US 2020/0035339 A1
(43) Pub. Date: Jan. 30, 2020

(54) BLOCKCHAIN SECURITY SYSTEM FOR SECURE RECORD ACCESS ACROSS MULTIPLE COMPUTER SYSTEMS

(71) Applicant: Orci Care Inc., Monmouth Junction, NJ (US)

(72) Inventors: Sri Ramesh Eevani, Monmouth Junction, NJ (US); Raghavendra Suresh, Bridgewater, NJ (US); Ravi Srinivasan, Franklin Lakes, NJ (US)

(21) Appl. No.: 16/272,588

(22) Filed: Feb. 11, 2019

Related U.S. Application Data
(60) Provisional application No. 62/703,748, filed on Jul. 26, 2018.

(51) Int. Cl. G16H 10/60; H04L 9/08; G06K 19/06; H04L 9/32; H04L 9/06

(52) U.S. Cl. CPC G16H 10/60; H04L 9/0819; G06K 19/06037; H04L 2209/38; H04L 9/3236; H04L 9/0637; H04L 9/0847

(57) ABSTRACT

A computer system and method that implements data security with the use of blockchain key encryption for healthcare records that can be accessed across multiple computer systems. The use of one or more blockchain ledgers allows the securing of data with different sets of keys between the computer platforms such that data can ultimately be secured only by the entity that controls the computer system with the healthcare records, with the security system itself only verifying and securing the user's identification data. The system therefore allows the healthcare provider to maintain mandated levels of data security for their stored records, but also allows a user of the system to provide access to other healthcare providers to the records for that user which are resident across multiple computer systems.

### CROSS-REFERENCE TO RELATED APPLICATION

This application claims the benefit of U.S. Provisional Patent Application Ser. No. 62/703,748, filed on Jul. 26, 2018, the entirety of which is hereby incorporated herein by this reference.

### BACKGROUND OF THE INVENTION

### 1. Field of the Invention

The present invention generally relates to data security systems and methods for securing data held in computer systems. More particularly, the present invention is for a blockchain security system that can provide secure record access and data transfer across multiple healthcare-related computer systems.

### 2. Description of the Related Art

A blockchain security method is a known way to provide secure transactions across the Internet. A “blockchain” is a continuously growing list of records, called “blocks,” that are linked through locational data on the network, and secured using cryptography. Each block normally contains a hash of the previous block, a timestamp, and some type of transaction data. Because of the linkage of blocks, the blockchain is highly-resistant to modification of the data held within a block. In one manner, the blockchain is an open and distributed ledger that can record transactions between parties efficiently and in a verifiable and permanent way, without the need for physical security of a data center or servers.

When setup as a distributed ledger, a blockchain is normally managed by a peer-to-peer network between computers that collectively adhere to a predetermined protocol for inter-computer communication and validating new blocks. Once the block is recorded and linked, the data in any given block cannot be altered retroactively without alteration of all subsequent blocks, which at a minimum would require at least a consensus of the majority of computers on in the blockchain network.

Because blockchains are secure and easily added to, they are well suited to the record events and other records management activities. They are also commonly used to serve as the public transaction ledger of cryptocurrencies, such as “Bitcoin.” The blockchain can thus be a decentralized and distributed public ledger that is used to record transactions allows the participants to verify and audit transactions. A blockchain can assign title rights, such as with a cryptocurrency, because it can detail the exchanges and provide a complete transactional record that compels offer and acceptance of the unit of cryptocurrency.

With respect to healthcare records and information stored electronically, a problem occurs in the security of the records, especially with respect to the privacy of the patient's data, which is the overriding concern and each individual healthcare computer system is consequently highly secured by itself. Even healthcare billing systems contain private medical data that is required to be secured at a minimum level by law. Thus, it is very difficult for a person to have healthcare information shared across multiple computer platforms such that the person can easily retrieve healthcare information in an electronic format, or have the various healthcare platforms share data across the platforms.

The problem of inter-communication between healthcare platforms becomes even more problematic when a person needs medical attention and is unable to easily provide access to medical records to a new provider. For example, for a medical emergency on vacation, a person may want to provide a foreign physician limited access to their medical records very quickly without compromising the overall security of the records. This can be even more difficult if the foreign healthcare provider is not a member of a trusted computer network or system that the person's home computer system will trust and interface with.

Further complications with the security of healthcare records can occur with the use of mobile applications that desire to access the secure healthcare data. Many mobile networks in the world are not considered sufficiently secure to allow remote access by a person. In the example of the need of foreign medical help, the only potential access to the healthcare computer system may in fact be a mobile device communicating from across the Internet.

### SUMMARY OF THE INVENTION

In overview, the present invention is for a system and method to implement data security for healthcare records being accessed across multiple computer systems with the use of blockchain key encryption. The use of one or more blockchain ledgers allows the securing of data with different sets of keys between the computer platforms such that data can ultimately be secured only by the entity that controls the computer system with the healthcare records, with the security system itself only verifying and securing the user's identification data.

In one embodiment, the system generates a private key internally for a user and stores it in a blockchain ledger. A user can then send a request to a healthcare provider to give them access to the healthcare records of the user that are stored with other providers. The request can be an e-mail containing a QR code or other standardized data code. The provider can then scan the code and the system will create a unique key for the provider that can be hashed with the private key held on the system for the user. The system will also store the key for the provider in another blockchain ledger and in this manner, the user key can provide access to the healthcare records as it will hash into the stored key of the provider, but does not by itself grant access to the healthcare records.

Through the use of blockchains in the present system, the computer hardware requirements for the healthcare record security, typically physically secure servers and data centers, can be mitigated and cloud-based computing resources utilized. The security level provided with the blockchains can meet almost all levels of mandated data security for healthcare records.

In one embodiment, the system can provide a limited access to healthcare records, potentially to a mobile device, and limit the access to the records and the ability to store the data. Such an embodiment is advantageous where access to the healthcare record is critical, such as a medical emergency in an unsafe area, but the data security needs to be maintained.

### BRIEF DESCRIPTION OF THE DRAWINGS

FIG. 1 is a representative diagram illustrating one embodiment of a blockchain security system that can provide secure access to health records across multiple computer systems.

FIG. 2 is a representative diagram illustrating one embodiment of the blockchain security system which uses multiple blockchains to provide secure access to a user of a mobile device to healthcare records across multiple computer systems.

FIG. 3 is a flowchart of one embodiment of a process for a user at a mobile device to register to use the blockchain security system.

FIG. 4 is a flow diagram of one embodiment of a process for a user of the blockchain security system to login into the system from an application at a mobile device.

FIG. 5 is a flow diagram of one embodiment of a process for a user of the blockchain security system to retrieve a lost password through an application resident at a mobile device.

FIG. 6 is a flow diagram of one embodiment of a process for a user of the blockchain security system to update their profile through an application resident at a mobile device.

FIG. 7 is a flow diagram of one embodiment of a process for a user of the blockchain security system to allow a healthcare provider already registered with the system to have secure access to the user's healthcare records resident on multiple computer systems.

FIG. 8 is a flow diagram of one embodiment of a process for a user of the blockchain security system to allow a healthcare provider that is not registered with the system to have secure access to the healthcare records of the user resident on multiple computer systems.

FIG. 9 is a flow diagram of one embodiment of a process for the user of the blockchain security system to view medical records at their mobile device that are sent from a hospital/medical system, with a notification to the user of the updating of medical records.

FIG. 10 is a flow diagram of one embodiment of a process for the user to have vitals data from a wearable device displayed to the user at a mobile device and storage of the data by the system.

FIG. 11 is a flow diagram of one embodiment of a process for a user of the blockchain security system to obtain a provider for a dependent.

FIG. 12 is a flow diagram of one embodiment of a process for a user of the blockchain security system create a profile for a dependent.

FIG. 13 is a flow diagram of one embodiment of a process for a user of the blockchain security system to get onto a provider waitlist for an appointment.

### DETAILED DESCRIPTION

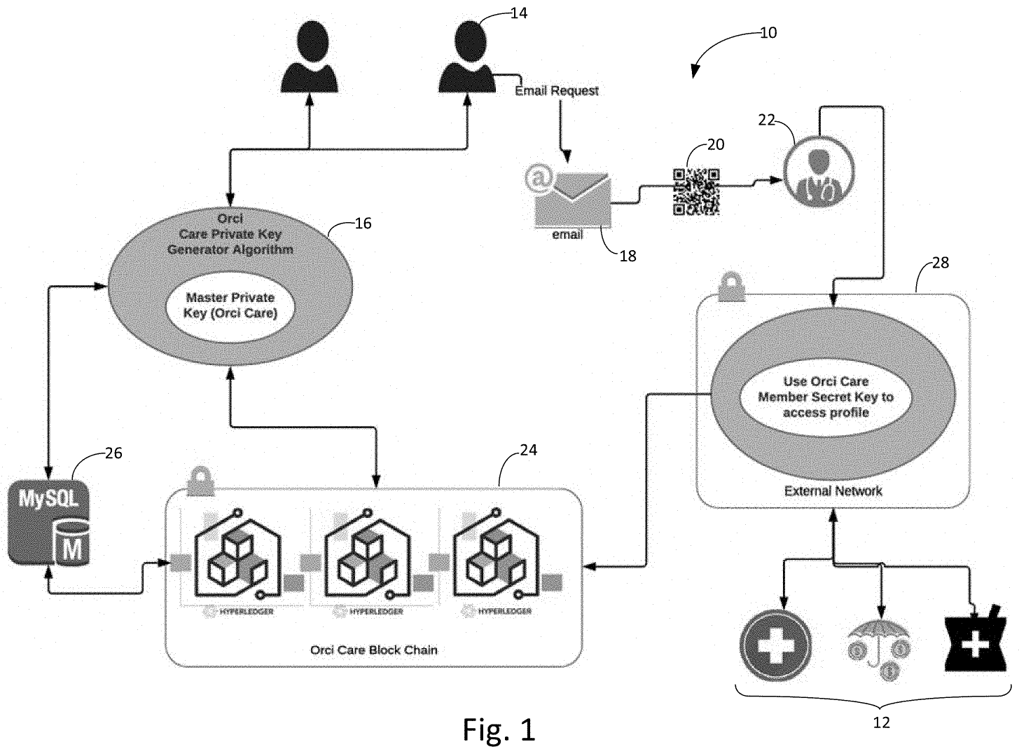

With reference to the figures in which like numeral represent like elements throughout, FIG. 1 is a representative diagram illustrating one embodiment of a blockchain security system 10 that can provide secure access to healthcare records 12 across multiple computer systems interconnected via the Internet. In this embodiment, users 14 can authorize a healthcare provider 22 to access the healthcare records 12 even if the healthcare provider 22 is not a registered provide of the system 10. The user 14 can send an e-mail request 18 to the provider 22 and the e-mail 18 can contain an access code, shown here as a QR code 20, that the provider 22 can scan to access user's 14 records.

In parallel with the generation of the QR code 20 for the provider 22, the system 10 can generate a private key for the user 14, as shown at process 16. A master private key can be generated from the system 10 along with a specific private key for the user 14 case at issue. In one example, a hash can be done with the username (known to the system 10) and the password created by the user 14. A general mathematical transform or algorithm to create a hash key can be used, such as a MOD algorithm, MD5, SHA1, or SHA2, or SHA-256 cryptographic hash algorithm. The generated keys are then stored in a blockchain system 24, such as Hyperledger Uledger, Multichain, or other Blockchain-as-a-service providers. Records of the key creation are then stored, such as in a SQL database 26. The key created in process 16 can then either be sent in the QR code 20 itself or the provider 22 can receive the key once they log into the system 10.

Once the provider 22 logs into the system 10 with the QR code 20 they will have access to the master key for that user 14 transaction to authorize the provider 22 to have access to the user 14 healthcare records 12 across the external system network 28. A comparison of the key with the blockchain system 24 will allow the provider 22 access to the external network 28 to obtain relevant healthcare records of the user 14. The master key can be for a one-time limited use and expire after a set amount of time, or the key can remain effective until withdrawn by the system 10 or the user 14.

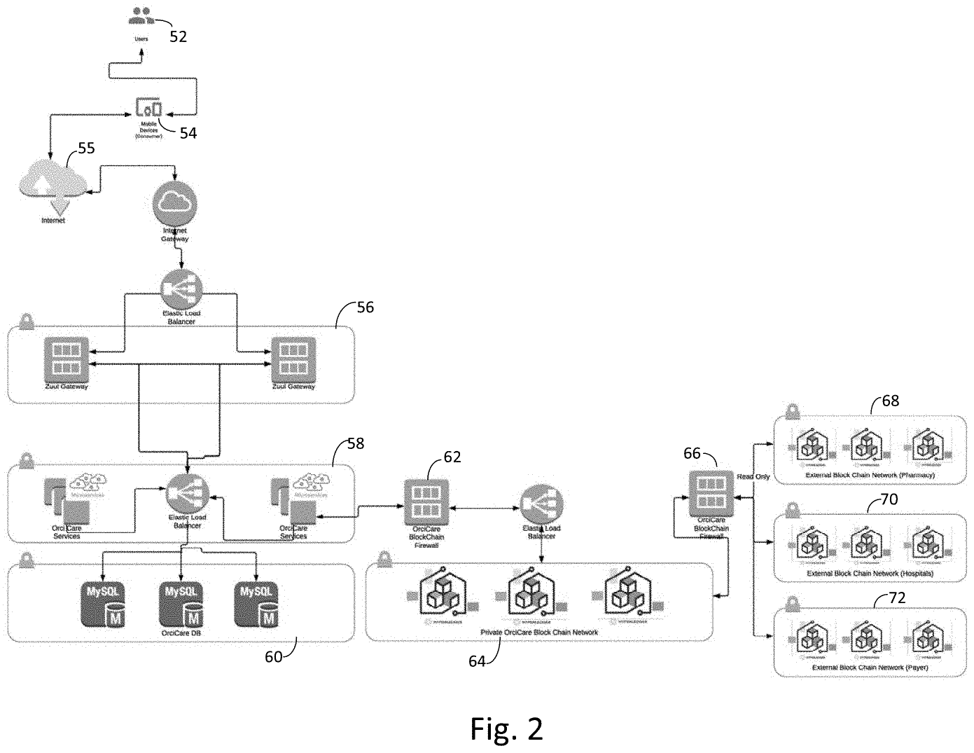

FIG. 2 is a representative diagram illustrating one embodiment of the blockchain security system 50 which uses multiple blockchains to provide secure access to a user 52 of a mobile device 54 to healthcare records across multiple computer systems 68,70,72. In this embodiment, the user 52 uses their mobile device 54 with an application resident thereon to access the system 50 through internet gateway 56 across the internet 15. An example of such a gateway is the Amazon Web Services portal. Internet access from mobile devices is also well-known and standardized. Once on the internet 55, the mobile device 54 can access the healthcare system 52 service level 58. The service level 58 is communication with the applicable record storage layer 60 as well as the blockchain ledger 64 for the system 50.

In this embodiment, there is a blockchain firewall 62 between the service level 58 and blockchain ledger 64. There is also an external blockchain firewall 66 between the blockchain ledger 64 and the provider blockchain ledgers, such as a pharmacy 68, hospital 70, or payor 72. In this embodiment, a blockchain ledger is created for each of the external networks such that those networks can provide their own layer of security in conjunction with the keys provided from the system 50. In this manner, paired key encryption, as is known in the art, can be used across multiple computer platforms with a high level of security.

In this embodiment, the system 50 is not even required to know what keys are generated at the provider blockchain ledgers 68,70,72 as it is not ultimately necessary for the system 50 to unencrypt anything sent from the providers. A hash comparison at the providers will allow decryption of the record. This configuration also lessens the resources needed by the system 50 to operate. The private blockchain ledger 64 will ensure that only the system 50 will have knowledge of the blocks of the ledger to verify user transactions within the system 50.

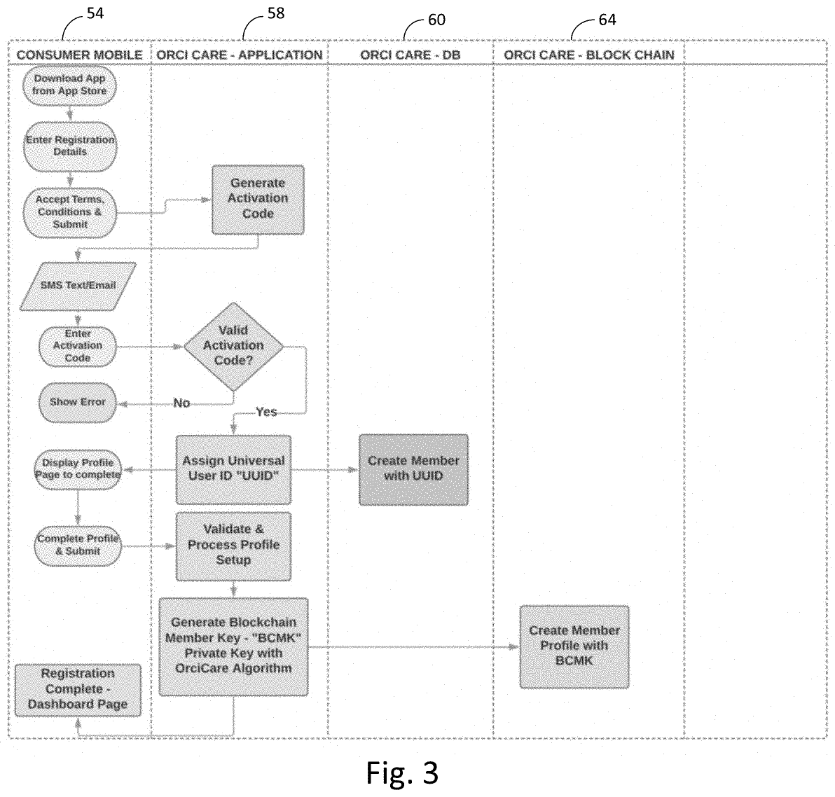

FIG. 3 is a flow diagram of one embodiment of a process for a user at a mobile device 54 to register a user for the blockchain security system 50 of FIG. 2. The mobile device 54 registers with the system 50 through a downloaded mobile application that becomes resident on the mobile device 54 and interface with the service level 58 of the system 58. Then the application interface at the service level 58 generates an activation code and returns it to the mobile device 54 and, in this embodiment, transmits the activation code with an SMS text to the mobile device 54.

Once the activation code is correctly activated at the mobile device 54, an interface is provided to the user 52 to finish inputting their profile information and a universal ID for the user 52 is created in the system 50 and stored in the record storage layer 60. Once the full profile is validated at the service level 58, the private key for that user 52 is then generated and stored in the blockchain ledger 64. Now the user 52 can access the system 52 to request that healthcare records be made accessible to other parties.

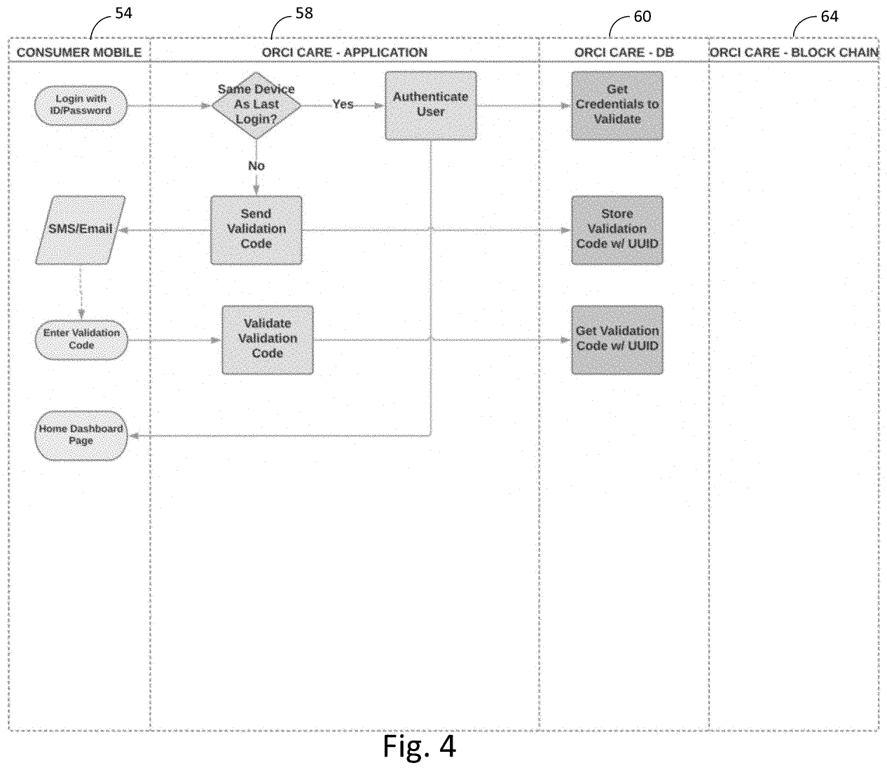

FIG. 4 is a flow diagram of one embodiment of a process for a user 52 of the blockchain security system to login into the system 50 from an application at a mobile device 54. The login ID and password known to the user 52 are input to the mobile device 54 application interface and the service level 58 attempts to authenticate the user 52 by retrieval of the credentials of the user 52 from the record storage layer 60. A validation code is then sent to the user 52 from the service level 58 and that code is also stored at the record storage layer 60 with respect to the specific login. The user 52 then enters the validation code into the application interface at the mobile device 54 and the service level 58 then receives the code and records proper validation of the user 52 at this login attempt at record storage layer 60.

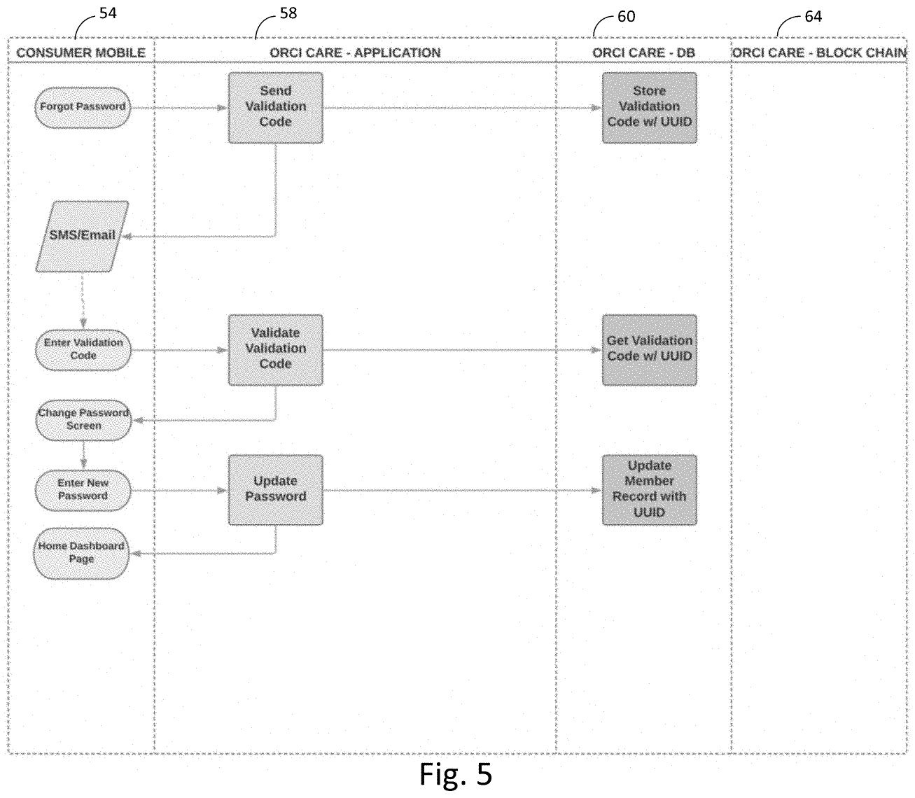

FIG. 5 is a flow diagram of one embodiment of a process for a user 52 of the blockchain security system 50 to retrieve a lost password through an application resident at a mobile device 54. A forgot password notice is sent from the mobile device 54 to the service level 58. The service level 58 attempts to authenticate the user 52 by retrieval of the credentials of the user 52 from the record storage layer 60. A validation code is then sent to the user 52 from the service level 58 and that code is also stored at the record storage layer 60 with respect to the specific login. The user 52 then enters the validation code into the application interface at the mobile device 54 and the service level 58 then receives the code and records proper validation of the user 52 at this login attempt at record storage layer 60, and then the service level 58 prompts the user 52 at the mobile device 54 to enter a new password. The password is then updated at the service level 58 and recorded in the record storage layer 60, and the user 52 is given access to the system 50.

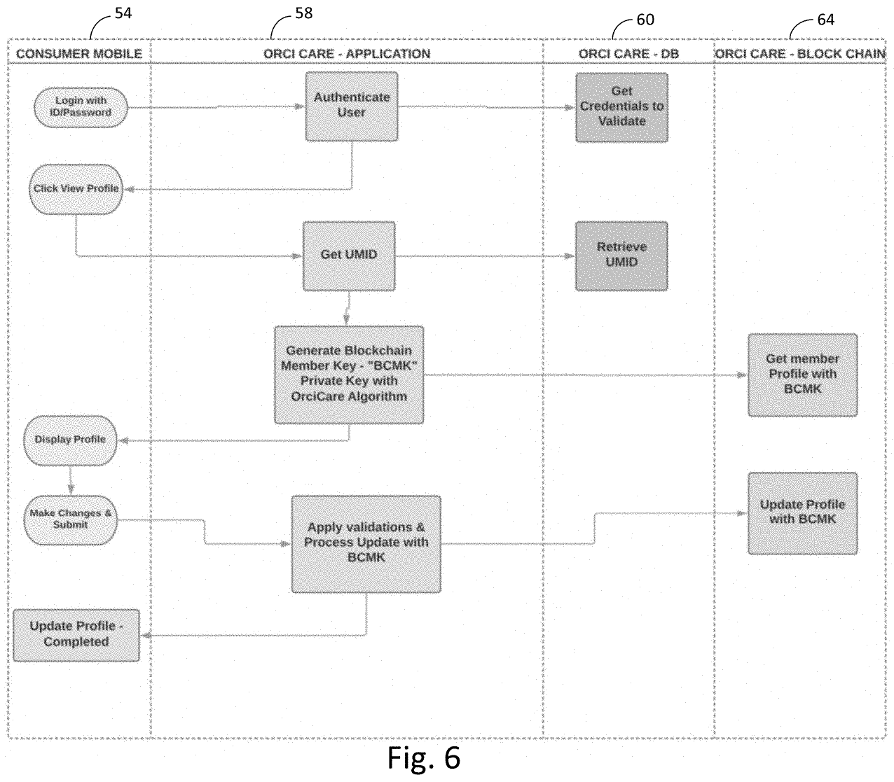

FIG. 6 is a flow diagram of one embodiment of a process for a user 52 of the blockchain security system 50 to update their profile through an application resident at a mobile device 54. The login ID and password known to the user 52 are input to the mobile device 54 application interface and the service level 58 attempts to authenticate the user 52 by retrieval of the credentials of the user 52 from the record storage layer 60. The user 52 then requests to view their profile and the service level 58 retrieves the user ID from the record storage level 60. The service level then generates a member key block for the user 52 and records that key in the blockchain ledger 64.

The service level 58 then permits the user 52 at the mobile device 54 to make changes to their profile and submit them back to the service level 58. The service level 58 then applies the changes and updates the private key held at the record storage layer 64 for the user 52 and notifies the user 52 that their profile has been updated.

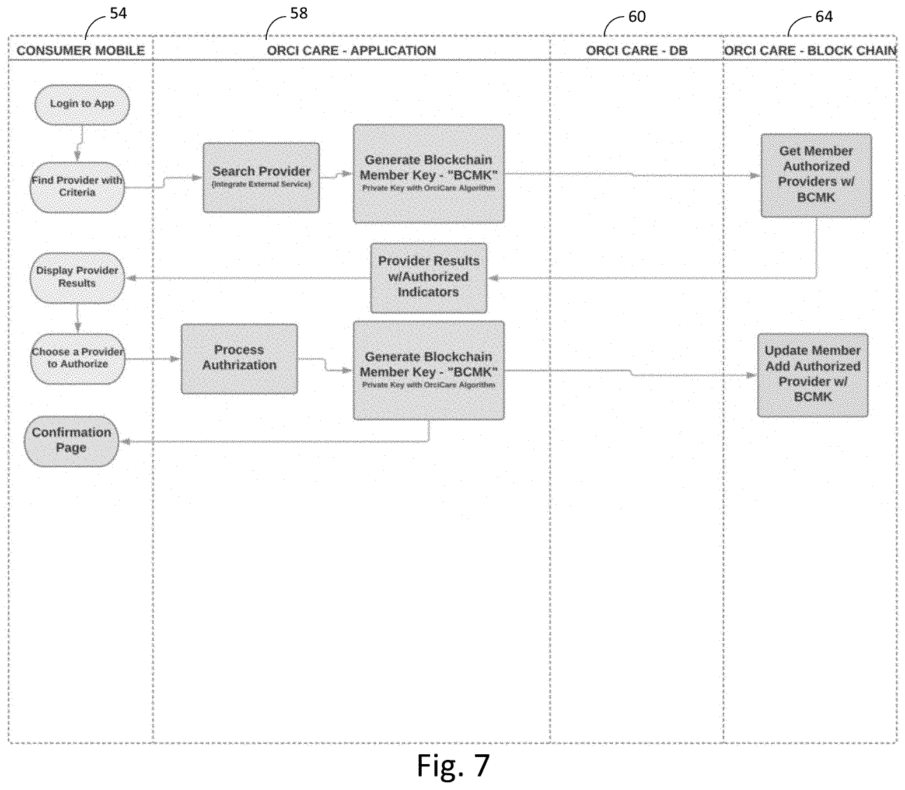

FIG. 7 is a flow diagram of one embodiment of a process for a user 52 of the blockchain security system 50 to allow a healthcare provider 12 already registered with the system 50 to have secure access to the healthcare records of the user 52 resident on multiple computer systems. The user 52 logs in to the application interface resident on the mobile device 54 and the user 52 searches for a provider based on some predetermined criteria. The service level 58 receives the search information for the provider then generates the private key for the user and searches the providers for the user 52 in the record storage level 64. Through this method, a layer of security can be placed between the record storage layer 60 and service level 58 whereby only the service level 58 would be able to validly access the search date for providers to that specific user 52.

The service level 58 then gathers the results for the search and sends them to the mobile device 54 for display. The user 52 can then choose a provider to authorize access to the healthcare records for the user 52 and transmit that choice to the service level 58. The service level 58 can then general the private key of the user 52 to identify the user 52 and then transmit the authorization of the provider to the record storage layer 60. The service level 58 can then confirm to the user 52 at the mobile device 54 that the provider has access the to the user's 52 healthcare records.

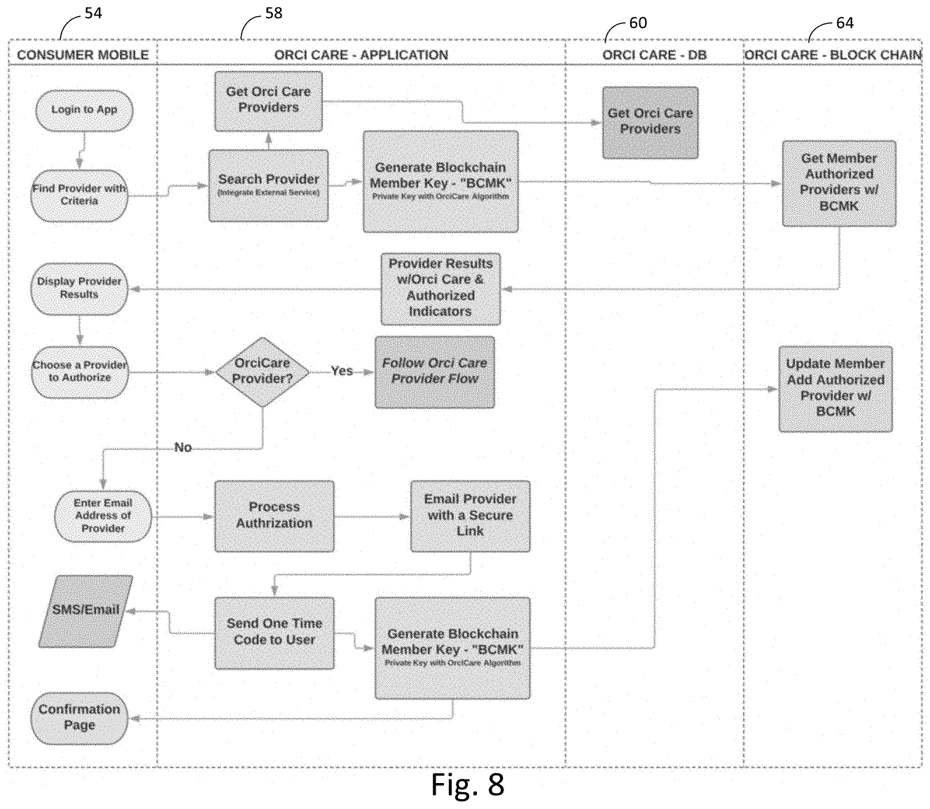

FIG. 8 is a flow diagram of one embodiment of a process for a user 52 of the blockchain security system 50 to allow a healthcare provider 12 that is not registered with the system 50 to have secure access to the healthcare records of the user 52 resident on multiple computer systems. The user 52 logins to the application interface resident on the mobile device 54 and the user 52 searches for a provider based on some predetermined criteria. The service level 58 receives the search information for the provider then first retrieves specific known provider 12 to the system 50 from retrieval of provider records from the record storage layer 60. The service level then generates the private key for the user 52 and searched the providers for the user 52 in the record storage level 64. The service level 58 then gathers the results for the search and sends them to the mobile device 54 for display, with providers 12 known to the system 50 identified in the sent information.

The user 52 can then choose a provider to authorize access to the healthcare records for the user 52 and transmit that choice to the service level 58. If the provider is known to the system 50, then the service level 58 follows the process of FIG. 7. Otherwise if the provider 12 is not known to the system 50, then the user 52 is requested to provide further information about the provider 12, such as an email address. The service layer 58 can then take the contact information for the unknown provider and email the provider a secure link to access the system 50.

Then a code is generated by the service level 58 and is sent to the user 52 at the mobile device and the user's private key is also generated such that the user 52 data can be identified in the blockchain ledger 64. The service level 58 can then general the private key of the user 52 to identify the user 52 and then transmit the authorization of the provider to the record storage layer 60. The service level 58 can then confirm to the user 52 at the mobile device 54 that the provider has access the to the user's 52 healthcare records.

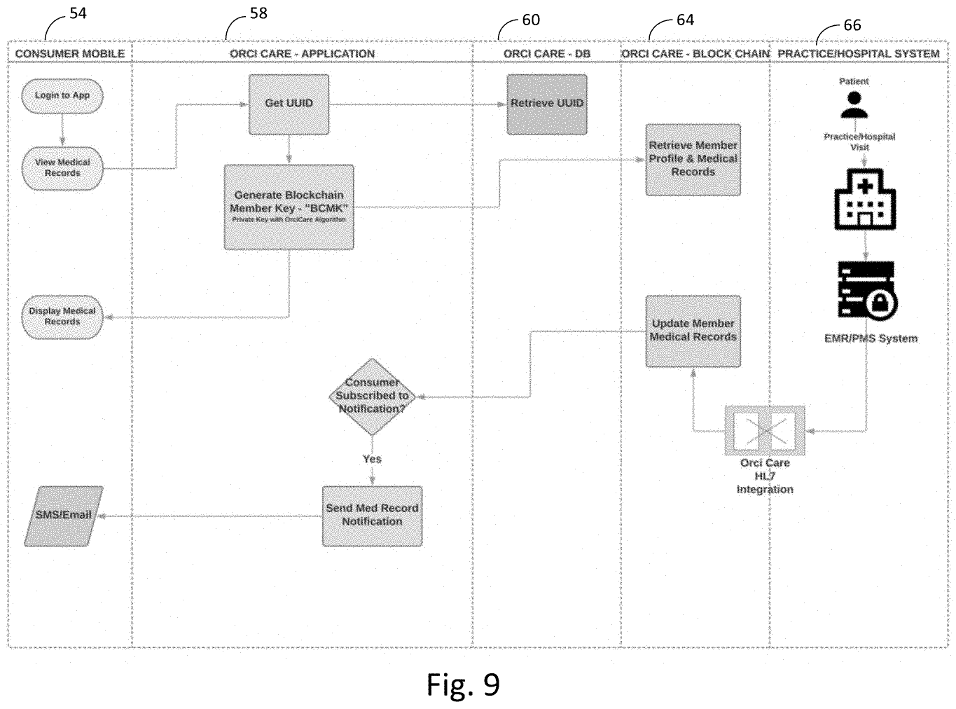

FIG. 9 is a flow diagram of one embodiment of a process for the user of the blockchain security system to view medical records at their mobile device 54 that are sent from a hospital/medical system 66, with a notification to the user of the updating of medical records. The user 54 logs in to the mobile application at the mobile device 54 and requests to view their medical records. The service level 58 then obtains the user's information from the record storage layer 60 and then generates the private key for the user. The service level 58 then retrieves the medical records that are secured via the blockchain ledger 64 which are then displayed to the user at the mobile device 54.

In this embodiment, the system 10 can update the medical records and notify the user. The user will have a medical visit which generates new information in the hospital system 66. The hospital system 66 then can generate a new record, potentially in the HL7 format when is then integrated and secured via the blockchain ledger 64. If the user is subscribed to received updates when new medical records are lodged, with the determination being made at service level 58, then notification of the new records is send out to the mobile device 54. The notification of the record update to the mobile device 54 can be a text, message, email, a voice message, or other form of communication.

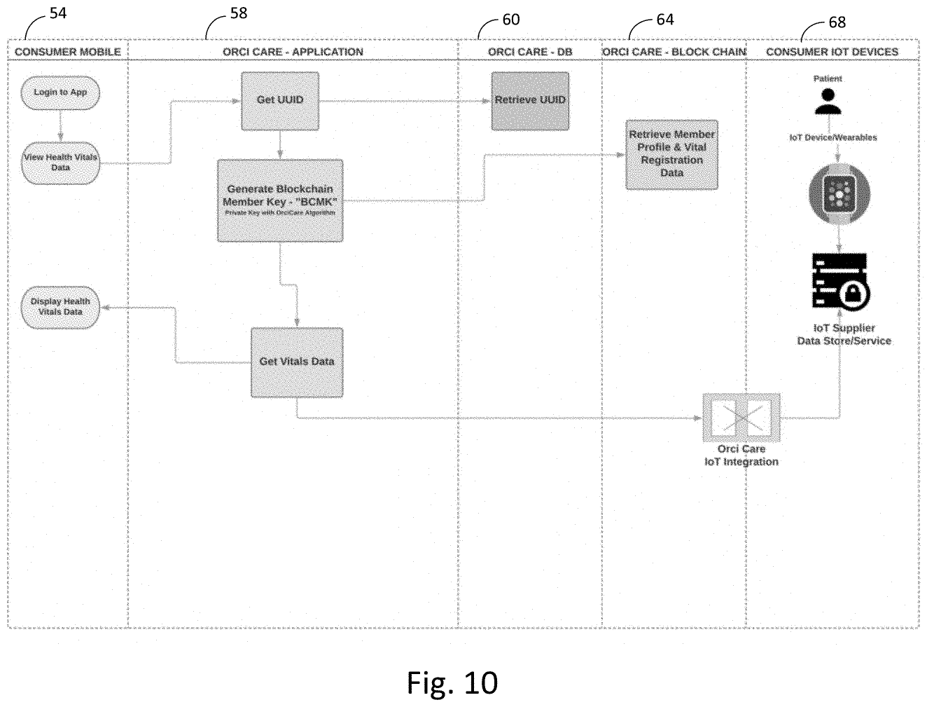

FIG. 10 is a flow diagram of one embodiment of a process for the user to have vitals data from a wearable device 68 displayed to the user at a mobile device 54 and storage of the data by the system 10. The user logs in to the mobile device 54 and requests to view their vitals data. The service level 58 then obtains the user's information from the record storage layer 60 and then generates the private key for the user. The service level 58 then retrieves the vital records data that are secured via the blockchain leger 64 which are then displayed to the user at the mobile device 54.

In this embodiment, the wearable device 68, which could also be any device from the “Internet of Things” (IOT) which will generate vitals data or other potential healthcare related data. The wearable device 68 will retrieve its data and/or live-feed the data into the system 10 at an interface. The interface can be in the HL7 format if desired. The service level 58 can then retrieve and display the vitals data to the user at the mobile device 54. The service level 58 can also store the vitals data in the blockchain ledger, or even send data to other entities storage, such as a data store or service for the wearable device 68.

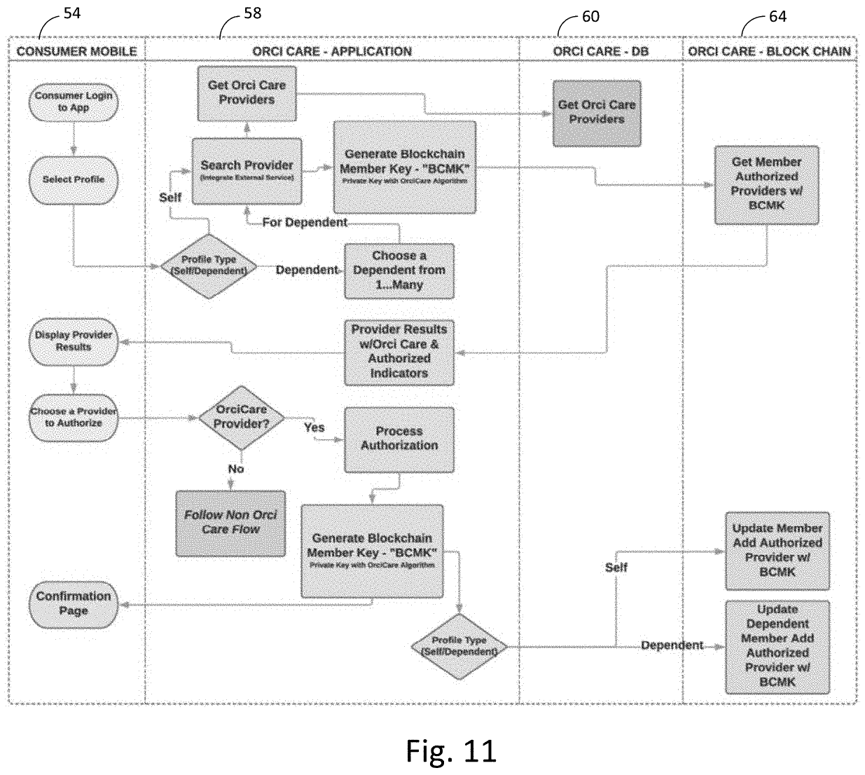

FIG. 11 is a flow diagram of one embodiment of a process for a user of the blockchain security system to obtain a provider for a dependent. The consumer mobile device 54 logs in to the system and the profile for the user of the system is determined at service level 58. The profile can be for the user herself, or for a dependent of that user. If the dependent is chosen at service level 58, then the provider(s) is obtained for the dependent and a blockchain member key is generated for that dependent and is stored in the blockchain ledger 64, and the providers for the dependent are retrieved from the record storage layer 60.

The provider results for the dependent are then displayed at the mobile device 54 and the user can select a provider of service at the service level 58. If the provider is chosen by the user a member key specific to the dependent is generated and stored at the blockchain ledger 64 and confirmation of the process is displayed to the user of the mobile device. The member key in the blockchain ledger 64 can be stored independently from any other record of the user, or can be linked with the user's records within the blockchain.

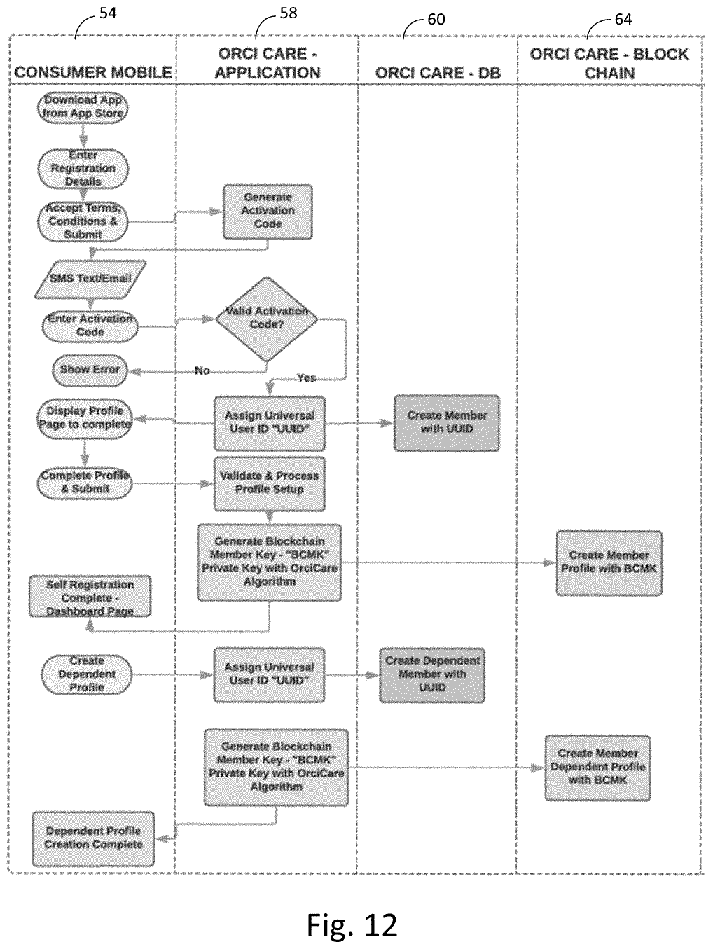

FIG. 12 is a flow diagram of one embodiment of a process for a user of the blockchain security system create a profile for a dependent. The process begins with the download of the application to the mobile device 54, and an activation code is sent from the service level 58 to the mobile device to provide a GUI for creation of a profile for the dependent. In a similar manner to initial creation of the user profile of FIG. 3, a unique member id is created for the dependent and stored in storage layer 60 and stored on the blockchain ledger 64.

Once instructed by the user at the mobile device 54, the service level 58 will assign a universal user ID for the dependent and store that identifier at record storage layer 60 and store the generated private key for the dependent on the blockchain ledger 64. The keys and records can be completed recorded separately from the user/main profile for the mobile device 54, or can be linked with that main profile as desired.

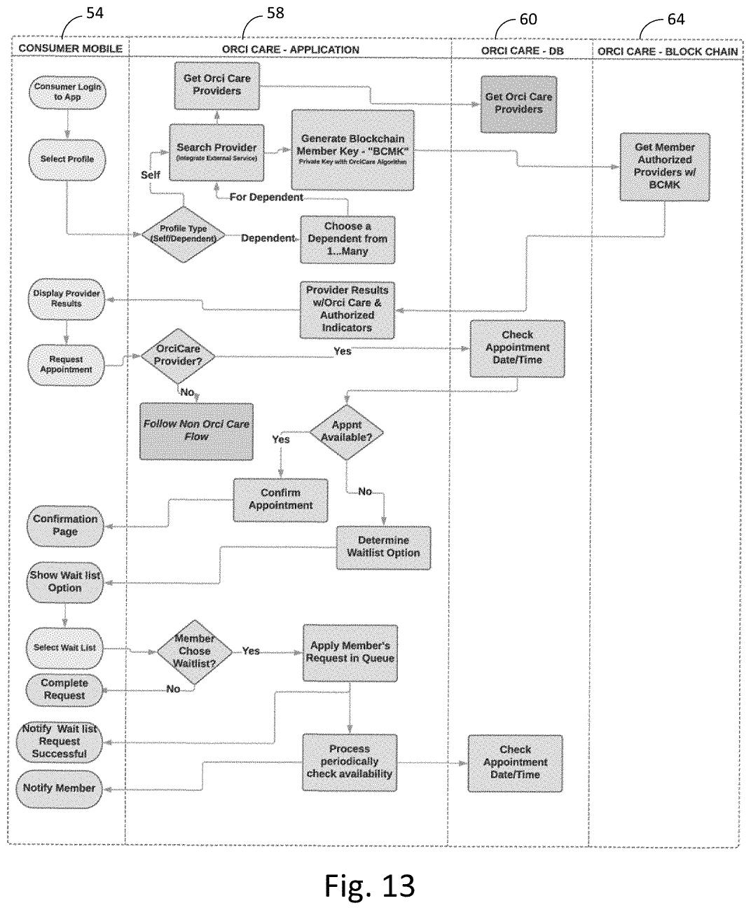

FIG. 13 is a flow diagram of one embodiment of a process for a user of the blockchain security system to get onto a provider waitlist for an appointment. The user at the mobile device 54, logs into the system at the service level 58 and will obtain providers for the user or dependent, in a manner similar to the process of FIG. 11. Once a provider is picked by the user at the mobile device 54, the user is then offered to request an appointment from the provider.

If the user desires to request an appointment at the mobile device 54, the service level 58 retrieves appointment times from the storage layer 60 and then determines if an appointment is available at the time requested by the user. In this embodiment, if the appointment time is available, a confirmation is sent to the user at the mobile device 54. If an appointment is not available, then a waitlist option is provided to the user at the mobile device 54.

If the waitlist option is not selected by the user at the mobile device 54, then the request is completed and the process is terminated. Otherwise, if the user wants to select the waitlist, the user is notified on the successful placement on the waitlist and is placed in the queue for appointments and the service level 58 periodically checks availability for the appointment. Upon the appointment being confirmed, the service level 58 notifies the user of the successful appointment and updates the storage level 60 accordingly with the appointment data.

The present invention has been described in several embodiments, and one of skill in the art is able to vary and change the elements of the systems and methods described herein without departing from the spirit and scope of the invention that is particularly set forth the in Claims.

What is claimed is:

1. A system of data security for healthcare records being accessed across multiple computer systems, comprising:
a user interface that receives a request from a user to allow access to records stored at healthcare providers having multiple computer systems;
a first key generator that generates a first key internally for a user and stores the first key in a first blockchain ledger;
a healthcare provider interface that receives requests from one or more healthcare providers that a user intends to give that healthcare provider access to the healthcare records of the user that are stored with other healthcare providers;
a second key generator that, upon receiving the request from a healthcare provider to grant access to the healthcare records of a user, creates a second key for the healthcare provider and stores that key, retrieves the first key stored for the user, and hashes the first key with the second key to create a unique access key for that healthcare provider, and the access key is stored in a second blockchain ledger; and
wherein the access key is selectively sent to the requesting healthcare provider such that the healthcare provider can access healthcare records of a user that are encrypted with the first key through comparison of the first key with the access key.

2. The system of claim 1, further comprising:
a QR code generator that selective provides a QR code to the user; and
wherein the healthcare provider scans the QR code as the request of the user to give that healthcare provider access to the healthcare records of that user stored at other healthcare providers.

3. The system of claim 2, wherein in the QR code contains the first key.

4. The system of claim 1, wherein the first key is created by performing a predetermined mathematical hash function on a username for a user and a password created by the user.

5. The system of claim 1, wherein the first blockchain ledger and second blockchain ledger are each a Hyperledger.

6. The system of claim 1, wherein the first key and second key are stored in a SQL database accessible to the system.

7. The system of claim 1, wherein the user interface further allows the user to register additional users into the system.

8. The system of claim 7, wherein the system further provides access to healthcare records at a mobile device through the use of the access key.

9. A method of providing data security for healthcare records being accessed across multiple computer systems, comprising the steps of:
receiving a request from a user interface that a user intends to provide access to a third party of healthcare records stored at one or more healthcare providers;
generating a first key for the user;
storing the first key in a first blockchain ledger;
receiving a request from a third party that a user intends to give that third party access to the healthcare records of the user that are stored with healthcare providers;
generating a second key upon receiving the request from a third party to grant access to the healthcare records of a user by retrieving the first key stored for the user;
storing the second key in a second blockchain ledger;
hashing the first key with the second key to create a unique access key for that third party;
storing the access key in a third blockchain ledger; and
selectively sending the access key to the requesting third party such that the third party can access healthcare records of a user that are encrypted with the first key through comparison of the first key with the access key.

10. The method of claim 9, further comprising the steps of:
generating a QR code for a user;
selectively sending a QR code to the user; and
upon a third party scanning the QR code, granting the third party access to the healthcare records of that user stored at other healthcare providers.

11. The method of claim 10, wherein the QR code is generated with the first key.

12. The method of claim 9, further comprising the step of generating the first key by performing a predetermined mathematical hash function on a username for a user and a password created by the user.

13. The method of claim 9, wherein the step of storing the first key, second key, and access key are storing each on a Hyperledger.

14. The method of claim 9, wherein the step of storing the first key, second key, and access key is storing each key in a SQL database.

15. The method of claim 9, wherein the step of receiving a request from a user interface is receiving a request from a mobile device interface.

16. The method of claim 15, further comprising the step of providing access to healthcare records at the mobile device for multiple users of the system.

17. A system for providing secure access of healthcare records at a mobile device, the healthcare records accessible across multiple computer systems, the system comprising:
a mobile device having a user interface that receives a request to provide access to a third party of healthcare records for the user;
a computer system that is accessible to the mobile device via a communication network;
wherein the mobile device sends the request from the user to the computer system; and
wherein, upon receipt if the request from the mobile device, the computer system:
generates a first key internally for a user and stores the first key in a first blockchain ledger;
upon receipt of a request from a third party that a user intends to give access to that third party of the healthcare records of the user that are stored with healthcare providers, the computer system generates a second key for the third party and stores the second key;
retrieves the first key stored for the user;
hashes the first key with the second key to create a unique access key for that third party and the access key is stored in a second blockchain ledger; and
selectively sends the access key to the requesting third party such that the healthcare provider can access healthcare records of a user that are encrypted with the first key through comparison of the first key with the access key.

18. The system of claim 17, further comprising the computer system generating a QR code and providing the QR code to the user; and
wherein the third party scans the QR code as the request of the user to give that third party access to the healthcare records of that user stored at other healthcare providers.

19. The system of claim 17, wherein the first key is created by performing a predetermined mathematical hash function on a username for a user and a password created by the user to access the system.

20. The system of claim 17, wherein the computer system further provides access to healthcare records at the mobile device.
